(19) United States
(12) Patent Application Publication
(10) Pub. No.: US 2011/0152895 A1
(43) Pub. Date: Jun. 23, 2011

(54) Apparatus for clamping an organ during surgery

(76) Inventors: Colin Nyuli, Vancouver (CA); Robert Nicholas Rohling, Vancouver (CA); Septimiu Edmund Salcudean, Vancouver (CA); Christopher Nguan, Vancouver (CA)

(21) Appl. No.: 12/972,488

(22) Filed: Dec. 19, 2010

Related U.S. Application Data
(60) Provisional application No. 61/282,145, filed on Dec. 22, 2009.

(51) Int. Cl. A61B 17/08

(52) U.S. Cl. 606/151

(57) ABSTRACT

This invention is a surgical clamp for the purpose of occluding the kidney or other organ and thereby obtaining a bloodless surgical field, while allowing the majority of the organ to remain normally perfused. Previous approaches are limited in ability to provide sufficient clamping force without damaging the oran. The invention comprises a first and second jaw, wherein the proximal ends of the two jaws, and the distal ends of the two jaws are connected by a strap, such that applying tension to the strap results in moving the relative positions of the jaws in a substantially parallel motion, which provide a clamping force to the organ. The jaws can be attached with a flexibly joint to a hollow shaft, such that a cable inside the shaft can be used to apply tension to the strap while maintaining the ability to pivot the jaws relative to the shaft.

### FIELD OF THE INVENTION

This invention relates generally to the occlusion of an organ or body conduit, and particularly to an apparatus for at least partially occluding an organ or body conduit during surgery.

### BACKGROUND OF THE INVENTION

The increasing trend towards employing minimally invasive surgical techniques in favor of conventional open surgical techniques is driven by benefits such as improved clinical outcomes with lower medical risks, shorter recovery times, and reduced costs to both hospitals and patients.

It is estimated that nearly 1,000 laparoscopic partial nephrectomies are performed in Canada every year, and likely more than 5,000 radical nephrectomies are performed. With an increasing number of incidentally detected small renal tumors detected in the general population, and with documented evidence for equivalent cancer control versus radical procedures, partial nephrectomy is becoming a standard of care in appropriate patients. Partial nephrectomy consists of removal of a portion of the kidney which harbours a renal tumor, with subsequent reconstruction and closure of the resection site defect to preserve the function of the remainder of the kidney. The intent is to maximize the amount of functioning kidney tissue to remain with the patient so as to promote overall long term renal functional capacity.

Laparoscopic partial nephrectomy requires great skill on the part of the surgeon due to the risks associated with control of bleeding during all stages of the surgery. Traditionally, bleeding is controlled through the use of scissor style clamps and forceps placed on the renal artery and in some cases vein and artery. However, these mechanisms leave the entire organ at risk for ischemia, and provides only a small window of time with which to carry out an operation without risking irreversible damage to the remaining renal tissue, generally less than thirty minutes.

A need exists for an apparatus with which to preferentially occlude the kidney or other organ and thereby obtain a bloodless surgical field, while allowing the majority of the organ to remain normally perfused during laparoscopic procedures. There have been attempts to preferentially occlude organs and body conduits during laparoscopic procedures in recent years. However, none of these approaches have proven to be entirely satisfactory. Problems include providing enough direct clamping force to fully occlude a preferable region of an organ such as the kidney or liver during a laparoscopic procedure, with minimal damage to the tissue.

### BRIEF SUMMARY OF THE INVENTION

It is an object of the invention to provide a solution to at least some of the deficiencies in the prior art.
1. One aspect of the invention is a surgical clamp comprising:
   a first jaw having a proximal end and a distal end;
   a second jaw having a proximal end and a distal end, wherein the proximal ends of the first and second jaw and the distal ends of the first and second jaw are operatively connected by a continuous strap;
   a means of applying tension to the strap such that when tension is applied, one or both of the jaws change position relative to each other but remain substantially parallel.
2. Another aspect of the invention is a surgical clamp of 1 further comprising a shaft pivotably connected with at least one jaw.
3. Another aspect of the invention is a surgical clamp of 2 further comprising a means of pivoting the jaws relative to the shaft.
4. Another aspect of the invention is a surgical clamp of 2 where the shaft is hollow and contains a means of applying tension to the strap.
5. Another aspect of the invention is a surgical clamp of 4 where the means of applying tension to the strap comprises applying tension to a cable in operable contact with the strap.
6. Another aspect of the invention is a surgical clamp of 1 wherein applying tension to the strap results in moving the jaws closer together.

### BRIEF DESCRIPTION OF DRAWINGS

FIG. 1A illustrates a diseased kidney and FIG. 1B illustrates the placement of a surgery apparatus on a diseased kidney.

FIG. 2 illustrates a side view of the top jaw and bottom jaw of the clamp of the surgery apparatus.

FIG. 3 illustrates the top jaw and bottom jaw of the clamp with an organ placed between the jaws.

FIG. 4 illustrates a laparoscopic surgery apparatus comprising a clamp, flexible neck assembly, endoscopic shaft, neck actuator for articulation, handle and clamp actuation knob.

FIG. 5 illustrates a side view of a clamp actuation knob, handle, neck actuator, nozzle and shaft.

FIG. 6 illustrates a side view of a clamp comprising an endoscopic shaft, flexible neck assembly, bottom jaw sleeve, top jaw, bottom jaw, and strap.

FIG. 7 illustrates a cross sectional view of a handle.

FIG. 8 illustrates a cross sectional view of a clamp excluding the bottom jaw sleeve.

FIG. 9 illustrates a handle showing internal threading.

FIG. 10 illustrates a clamp actuation knob.

### DETAILED DESCRIPTION OF THE INVENTION

Directional terms such as “proximal”, “distal”, “left”, “top” and “bottom” are used in the following description to indicate relative reference only, and should not impose any limitations on how any apparatus or components thereof are to be manufactured or positioned during use.

Embodiments of the invention described herein relate to a surgery apparatus for mechanically compressing organs during minimally invasive surgeries in order to at least partially occlude fluid flow into, out of, or within an organ. In one exemplary embodiment, the surgery apparatus comprises a clamp which comprises two rigid, substantially parallel opposed jaws configured to grasp organ tissue and apply clamping force so as to at least partially occlude the blood flow of an organ. In one aspect of the invention, the jaws are coupled by a strap such that when the strap is actuated, the jaws move closer together. The means of actuating the strap can be many known in the art, and include attaching a cable to the strap and pulling the cable.

In one embodiment, the clamp comprises two rigid, opposed jaws (a top and bottom jaw) coupled to each other such that they remain substantially parallel. The jaws may be coupled by a strap composed of a material of high strength and flexibility. The bottom jaw has a proximal end, a distal end, and may include a serrated gripping pattern etched onto or attached to at least part of its surface. The top jaw has a proximal end, a distal end, and may also include a serrated gripping pattern etched onto or attached to at least part of its surface. Preferably, one jaw moves relative to the other jaw and the two jaws remain in a substantially parallel configuration during the movement. At least one of the jaws moves with respect to the other to represent at least two positions: closed, where the jaws are closer relative to each other, and open, where the jaws are further apart relative to each other. In the open position, a material can be positioned in the space between the jaws, where the material includes an organ, blood vessel or other body material. Other positions are possible, where the jaws are at different distances relative to each other but remain substantially parallel.

In some cases, it may be preferable to have jaws that are substantially planar or of greater width. Some embodiments of the invention may thus have jaws that resemble plates. In other embodiments, it may be desirable to have jaws that have some degree of flexibility to minimize trauma to the organ or tissue being clamped. While the jaws in these embodiments may have some ability to bend around the contours of an organ, it should be understood that the jaws still remain substantially parallel with respect to each other.

The top and bottom jaws are coupled together using various actuating assemblies. An actuating assembly may comprise a strap coupled to at least one of the jaws, a cable or other device that pulls the strap, and a device for pulling the cable. In one exemplary embodiment of a clamp actuation assembly, a strap is fastened to both the proximal and distal ends of the top jaw and coupled to the bottom jaw through a slot located at the distal end of the bottom jaw, and at a proximal insertion located at the proximal end of the bottom jaw. The strap may form a loop which couples the top and bottom jaws and when pulled, it engages the top jaw, allowing for clamp actuation and positioning of the jaws such that they are closer together. The strap may be pulled by various mechanisms, including by a cable. In other embodiments of the actuating assembly, the strap may not form a loop but may consist of a pair of strap sections, one strap section extending from the distal end of the top jaw passing to and then along the bottom of the bottom jaw and into the sleeve (handle) and the other section extending from the proximal end of the top jaw passes around a rotatable spacer, and into the sleeve where both these ends are attached to the cable to be equally moved by movement of the cable, i.e. to close the clamp. Preferably, as one or both jaws move they remain substantially parallel.

In other embodiments, the clamp actuation assembly may include other mechanisms for moving the jaws relative to each other, such as a spring, ratcheting levers or a sliding locking handle component. Other actuation assemblies are possible that are known in the art, and preferred assemblies will allow an operator to induce a variable clamping force to tissue between the jaws of the clamp. In certain embodiments, the clamp actuation assembly can be triggered manually to induce a clamping force, but other embodiments are possible where the clamp actuation assembly is activated automatically or by a surgical robot, and may be based on feedback received by a measurement of the clamping force or measurement of tissue. For example, a system operating the surgery apparatus may be a surgical robot that measures the clamping force in the tissue and adjusts the clamp actuation assembly accordingly to attain a preset force level. Or, the surgical robot may measure the blood flow in an organ while simultaneously inducing a clamping force through the clamp actuation assembly and adjust the clamping force accordingly to obtain a sufficiently low level of blood flow, or a preset blood flow level.

The surgery apparatus may also comprise an endoscopic shaft, a handle, a flexible neck assembly to allow movement of the clamp relative to the endoscopic shaft, and an articulating assembly to control the movement of the clamp relative to the endoscopic shaft.

In preferred embodiments of the surgery apparatus comprising an endoscopic shaft, the endoscopic shaft is in operable connect with the clamp and allows the clamp to be inserted into a body cavity. The endoscopic shaft has a proximal end, a distal end, and may be mounted to the proximal end of the bottom jaw of the clamp. The endoscopic shaft may be mounted to the clamp through a flexible neck assembly and/or a bottom jaw sleeve. The flexible neck assembly allows for angulation of the clamp relative to the endoscopic shaft during surgery. A bottom jaw sleeve provides structural support and prevents tissues from entering the endoscopic shaft and may be included in the apparatus. The endoscopic shaft may house a cable or other device coupled to the strap, where the cable or device is pulled to actuate the jaws of the clamp via the strap.

The surgery apparatus may also comprise an articulation assembly to move and hold the clamp relative to the endoscopic shaft by flexing the neck to a desired angle relative to the shaft. In one embodiment, the articulation assembly is a power screw that, when rotated, transmits energy through an elongated flexible transmission band assembly to induce movement, such as rotational, in the clamp through the flexible neck assembly. In other embodiments, the articulation assembly may include other mechanisms for moving the clamp relative to the endoscopic shaft, such as a spring, ratcheting levers or a sliding locking handle component. Other articulation assemblies are possible that are known in the art, and preferred assemblies will allow an operator to induce a variable change in the position, such as the angle relative to the shaft, of the clamp without moving the shaft, which is preferable during non-invasive surgery. In certain embodiments, the articulation assembly can be triggered by an operator to adjust the position of the clamp, but other embodiments are possible where the articulation assembly is activated automatically or by a surgical robot, and may be based on feedback received by a measurement the surgical field, such as through ultrasound imaging or a laparoscopic camera.

In certain embodiments of the invention, the surgery apparatus allows the clamping of the jaws and the articulation of the neck to be controlled independently.

The surgery apparatus may also comprise a handle for grasping the apparatus during laparoscopic surgery, which may be connected to the endoscopic shaft directly or to a nozzle containing a neck actuator, which is connected to the endoscopic shaft. The neck actuator is part of the articulation assembly. The handle may have a hollow body through which a cable or other device connected to the strap of the clamp is positioned. The handle may contain internal threads which mate with a clamp actuation knob that acts as a power screw to pull a cable or other device, which in turn pulls the strap of the clamp. In this case, the clamp actuation knob and the cable (or other device) are part of the clamp actuation assembly for moving the jaws of the clamp relative to each other.

In one embodiment, the clamp actuation knob has a threaded distal end, and a proximal end of larger diameter than the distal end, with knurled texture for gripping and twisting, and there is an inner hollow channel extending from the distal end to the proximal end. The proximal end of the handle may mate with the clamp actuation knob in rotational contact.

In one example of the surgery apparatus, the clamp actuation assembly comprises a cable, a strap and a clamp actuation knob. The cable runs from the strap coupled to the top jaw to the proximal end of the clamp actuation knob housed in the handle. The cable may be coupled to a bushing which is housed in the proximal end of the clamp actuation knob and provides for sliding contact and force transmission between the cable and the clamp actuation knob. The bushing may be composed of a variety of materials, including Teflon, rubber or metal.

In the embodiments where a power screw and a clamp actuation knob are used for the clamp actuation assembly, a fine control is provided by the torque-to-force multiplication factor of the screw pitch. For example, a torque-to-force multiplication factor of 10 is easily achieved between the clamp actuation knob and cable, while at the same time providing for linear distance increments between top jaw and bottom jaw of only 2 mm per full rotation. When a power screw and clamp actuation knob are used in certain embodiments, the power screw may be threaded with a pitch to provide locking so as to disable any back-driving caused by pressure experienced between the top jaw and the bottom jaw. Generally, multiple mechanisms for preventing the jaws of the clamp from separating are possible and will become apparent for inclusion in the surgery apparatus. Mechanisms for preventing the strap of the clamp from loosening and the jaws separating include the inclusion of a pin and tooth lock on the clamp actuation knob, an axial friction lock on the clamp actuation knob or along the shaft, or pins for insertion into holes along the shaft. The surgery apparatus may include multiple mechanisms for preventing the strap of the clamp from loosening, and may include a mechanism of emergency release for rapid separation of the jaws of the clamp, such as by cutting or releasing the strap or cable from their attachment points.

The clamp actuation assembly for the clamp may cause the jaws of the clamp to be brought closer together when the strap of the clamp is pulled, such as by turning a clamp actuation knob. Some actuation assemblies may not result in the jaws moving further apart if the clamp actuation assembly is driven in reverse, such as by turning a clamp actuation knob in the reverse direction. In such cases, the top jaw and bottom jaw may be brought apart by the use of forceps or any other such grasping tool known in the art. An example of a mechanism for bringing the jaws of the clamp apart is by allowing the jaws to be constructed of flexible material with a preformed curved shape that is flattened by clamping the jaws together, the clamp can be inserted through the trocar; subsequently releasing the clamping force allows the jaws to spring apart as they re-attain their naturally curved shape.

Preferred embodiments of the invention will provide approximately uniform occluding force along the length of the top and bottom jaws due to the ability of the jaws to remain substantially parallel throughout movement relative to each other.

Certain materials are preferable for the composition of the components of the surgery apparatus. The strap is ideally composed of a material of high strength and flexibility, such Nitinol. The cable or material for actuating the jaws of the clamp is preferably made from a strong material, such as steel, nylon or other plastic or metal materials, including composites.

The surgery apparatus may be used in many ways to at least partially occlude fluid flow. It will be apparent that the surgery apparatus can be used for any procedure that requires the occlusion of blood flow to at least part of an organ or other body part such as the vasculature. The surgery apparatus is particularly suited to minimally invasive surgery, where the surgery is performed by inserting instruments through small incisions in the body. However, the surgery apparatus can also be used for any type of surgery including conventional surgery with a large incision. Examples of surgeries, either minimally invasive (e.g. laparoscopic, robotic or endoscopic) or otherwise, during which the surgery apparatus can be used include, but are not limited to, resections, partial resections, ablations or cryoablations of the kidney, liver, spleen, pancreas, adrenal glands, lungs, heart, vasculature, musculature, small and large bowels and other internal organs.

In other embodiments, the jaws of the surgical apparatus can be used to perform ablation of tissue, and may contain electrodes. The jaws can effect ablation on tissue in contact with surface of one or both jaws by different mechanisms including, but not limited to, radiofrequency energy and cryogenics.

The following descriptions and figures should serve to provide depictions and illustrations of embodiments of the invention and should in no way restrict the scope of the invention.

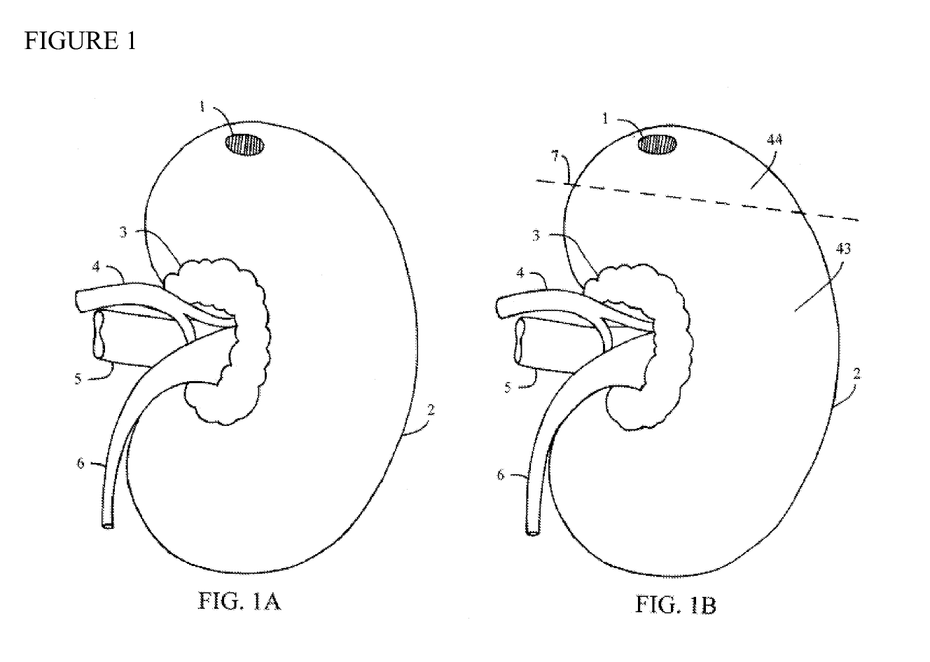

To illustrate an example of the surgery apparatus for clamping an organ, FIG. 1 depicts a kidney that is compressed by a clamp to at least partially occlude blood flow. In FIG. 1A, a schematic of a kidney 2 includes a tumor 1 and vasculature, which includes a renal artery 4, a renal vein 5, a ureter 6 and a hilar fat pad 3. As shown in FIG. 1B, a clamping force is applied along a clamp-placement line 7 such that the kidney 2 now comprises a normally perfused portion 43 and an occluded portion 44, in which the blood flow is at least partially occluded, and from which the tumor 1 can be resected.

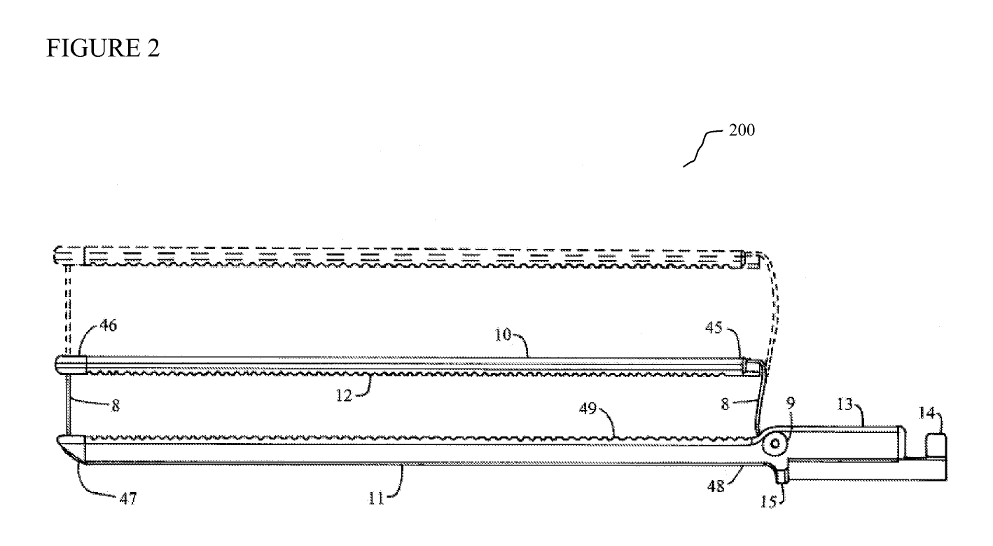

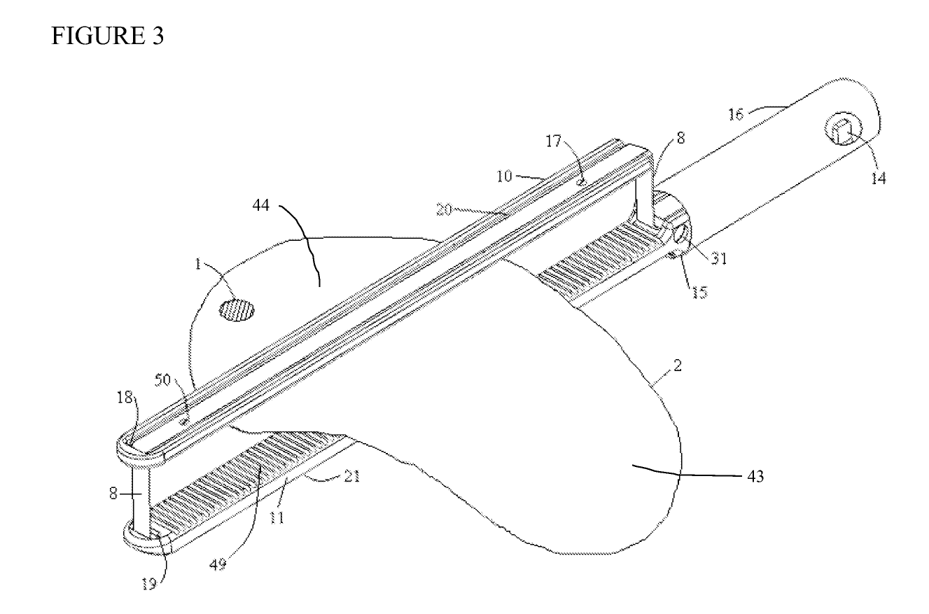

FIG. 2 schematically illustrates an embodiment of the clamp 200 by demonstrating a side view of the clamping mechanism composed of top jaw 10 and bottom jaw 11. Dotted lines represent an alternative positioning of the top jaw 10 relative to the bottom jaw 11, where the top jaw is at a further distance from the bottom jaw 11 but remains substantially parallel. In the dotted line configuration, a strap 8 is adjusted so that it extends to allow the jaws to remain further apart, but can be also adjusted to move the jaws closer together and substantially parallel. The top jaw 10 has a rectangular configuration with a serrated gripping surface 12 extending between a proximal end 45 and a distal end 46. The bottom jaw 11 has a rectangular configuration with a serrated gripping surface 49 extending between a proximal end 48 and a distal end 47. The bottom jaw 11 includes a support-bolt hole 9 which houses a support bolt (see 31 of FIG. 3) that retains and applies resistive force to a strap 8 that, in this case, forms a continuous loop between top jaw 10 and bottom jaw 11. By pulling both ends of strap 8 an equal distance into the bottom jaw sleeve body 13, then the strap lengths between the adjacent ends of the top jaw 10 and bottom jaw 11 will be maintained equal, and the top jaw 10 and bottom jaw 11 will remain substantially parallel, and will be moved toward each other to close the clamp. Other configurations are possible, such as where the strap 8 does not form a continuous loop but is fixed to the distal end 47 of the bottom jaw 11. The proximal end 48 of the bottom jaw 11 includes the bottom jaw sleeve lip 15 against which the bottom jaw sleeve 16 (shown in FIG. 3) abuts, in order to provide support for the clamping mechanism. The bottom jaw sleeve body 13 can be cylindrical, allowing for relative coupling to the cylindrical bottom jaw sleeve 16, and terminating in two bottom jaw mating posts 14.

FIG. 3 schematically illustrates an embodiment of the clamp 200 used for occluding an organ, such as a kidney as depicted. In this depiction, the clamp consists of top jaw 10 and bottom jaw 11 in contact with a kidney 2 for the purpose of occluding blood flow to a part of the kidney 2 containing a tumor 1 (the occluded portion 44) while maintaining blood flow to the remaining part of the kidney 2 (the perfused portion 43). In this embodiment, additional features of the top jaw 10 include two screw holes (17, 50) for fixing the strap 8 into the groove 20 and thus to the top jaw 10. The strap passes through the slot 18 in the top jaw and is directed to the disal end of the bottom jaw 11. In this embodiment, additional features of the bottom jaw 11 include a bottom jaw strap slot 19 which directs the strap 8 bottom jaw strap groove 21 through which the strap moves to the sleeve body 13. A support-bolt and nut 31 may be fastened to a support-bolt hole 9 in the bottom jaw 11 and the strap 8 passing between the proximal ends of the jaws 10 and 11 passes around the nut 31 and the two ends of the strap exteding from the jaw 10 are brought together for coupling to the cable 33 as will be described below. In this depiction, a bottom jaw sleeve 16 is shown abutted against a bottom jaw sleeve lip 15.

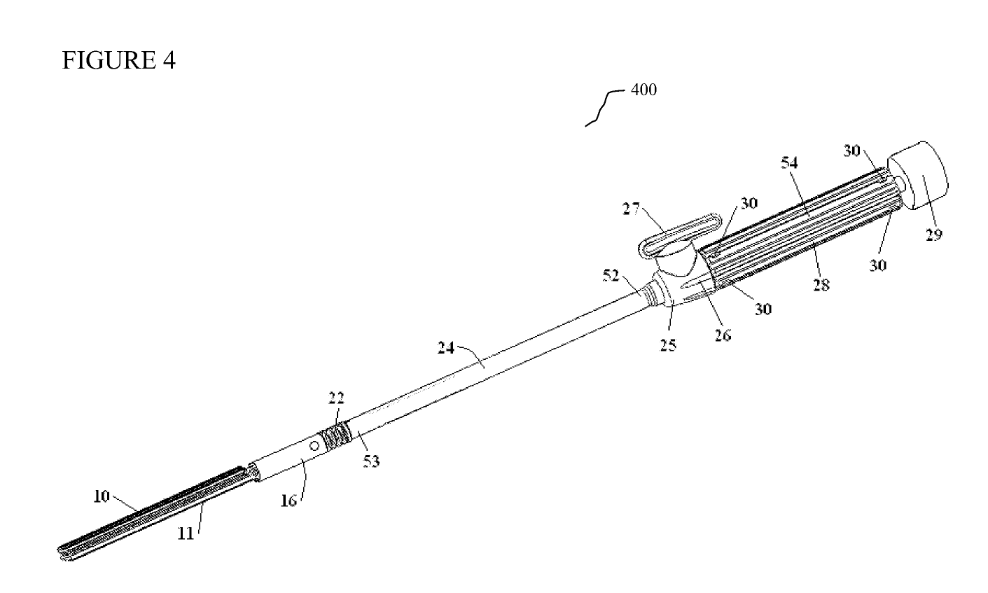

FIG. 4 illustrates an embodiment of the surgery apparatus 400 that includes a clamp 200, flexible neck assembly 22, endoscopic shaft 24, handle 28, neck actuator 27 and clamp actuation knob 29. In this depiction, the top jaw 10 sits in a closed configuration with respect to the bottom jaw 11 in order to be inserted through a trocar into an opening of the body, such as through the peritoneum of the abdomen, for laparoscopic surgery. A cylindrical bottom jaw sleeve 16 is mounted between the flexible neck assembly 22 and the top jaw 10 and bottom jaw 11 in order to provide structural support and prevent tissues from entering the endoscopic shaft 24. The flexible neck assembly 22 allows for rotational and linear articulation of the clamp. The endoscopic shaft 24 is of cylindrical geometry for the purpose of trocar insertion and extends between a proximal end 52 and a distal end 53. The proximal end 52 of the endoscopic shaft 24 is externally and concentrically coupled to a nozzle 25 that mates the endoscopic shaft 24 and the handle 28.

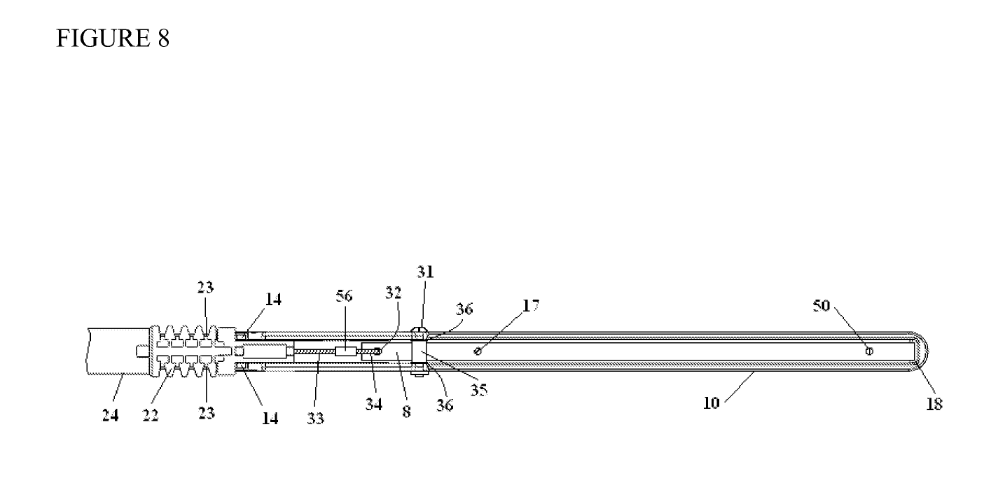

The articulation assembly, in the depiction in FIG. 4, comprises the nozzle 25, the neck actuator 27, the flexible neck assembly 22 and the flexible transmission band assembly 23 (shown in FIG. 8) in order to induce angulation of the clamp relative to the endoscopic shaft 24. The nozzle 25 may have a textured grip 26 in order to allow for easy rotation, which in turn axially rotates the clamp 200 and therefore the jaws (10, 11). When the neck actuator 27 is manipulated, rotary motion is transmitted by a pinion internal to the neck actuator 27 along an elongated flexible transmission band assembly (see 23 of FIG. 8) housed inside the endoscopic shaft 24, through the flexible neck assembly 22 and directly to the bottom jaw mating posts 14. The articulation assembly can be constructed using various mechanisms known in the art.

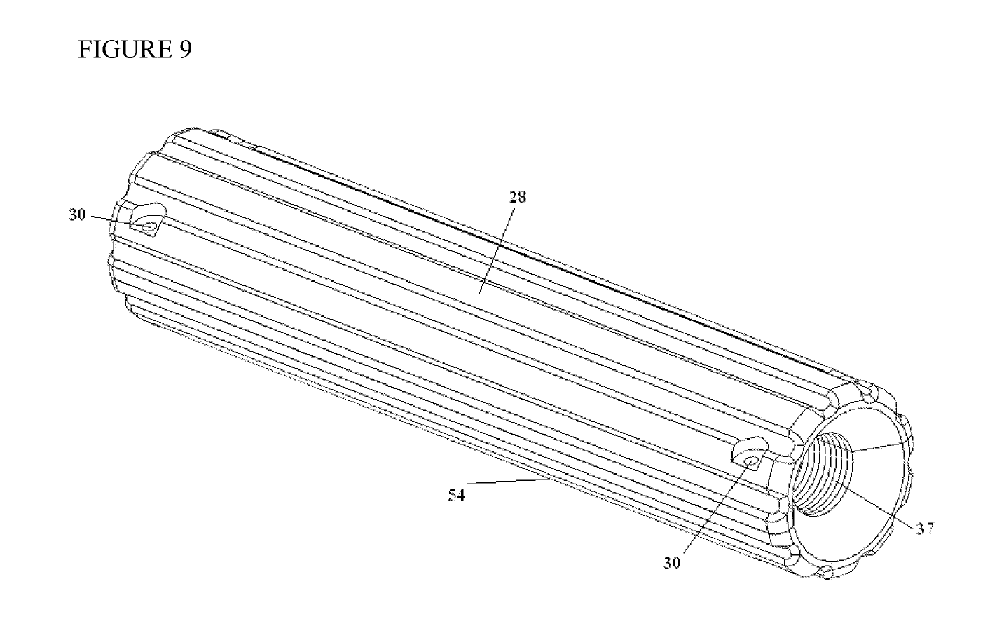

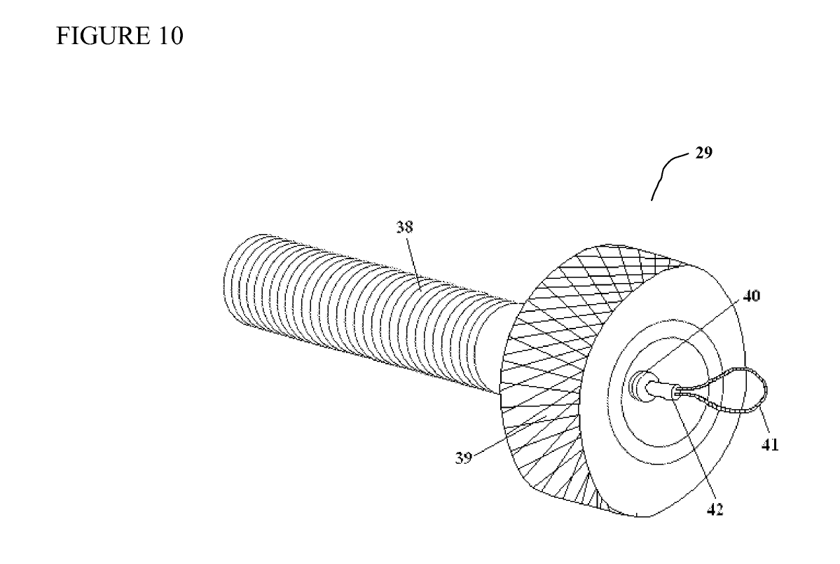

FIG. 4 also schematically illustrates an embodiment of the assembly of a handle (composed of 28 and 54) and clamp actuation knob 29. The clamp actuation knob 29, in combination with the cable (see 33 of FIG. 8), comprise the clamp actuation assembly for moving at least one jaw of the clamp relative to the other. In this depiction, a linear handle male section 28 mates by threaded fasteners which are placed through four peripherally placed handle bolt holes 30 to the linear handle female section 54. Both the linear handle male section 28 and linear handle female section 54 may be internally threaded (see 37 of FIG. 9) in order to accept the externally threaded section (see 38 of FIG. 10) of the clamp actuation knob 29 when fastened together. Both the linear handle male section 28 and linear handle female section 54 may be externally textured by linear handle grip texture 55 in order to increase friction between a hand and the handle. By turning the clamp actuation knob 29, an internally fastened cable (see 33 of FIG. 8) is pulled by the knob's axial motion and provides actuation to the top jaw 10. It should be noted that the clamp actuation knob 29 may only cause the top jaw 10 to be brought into closer proximity with the bottom jaw 11, and may not cause the top jaw 10 and the bottom jaw 11 to be brought apart if driven in the reverse direction (clockwise). In such cases, the top jaw 10 and bottom jaw 11 may be brought apart by the use of forceps or other methods and mechanisms.

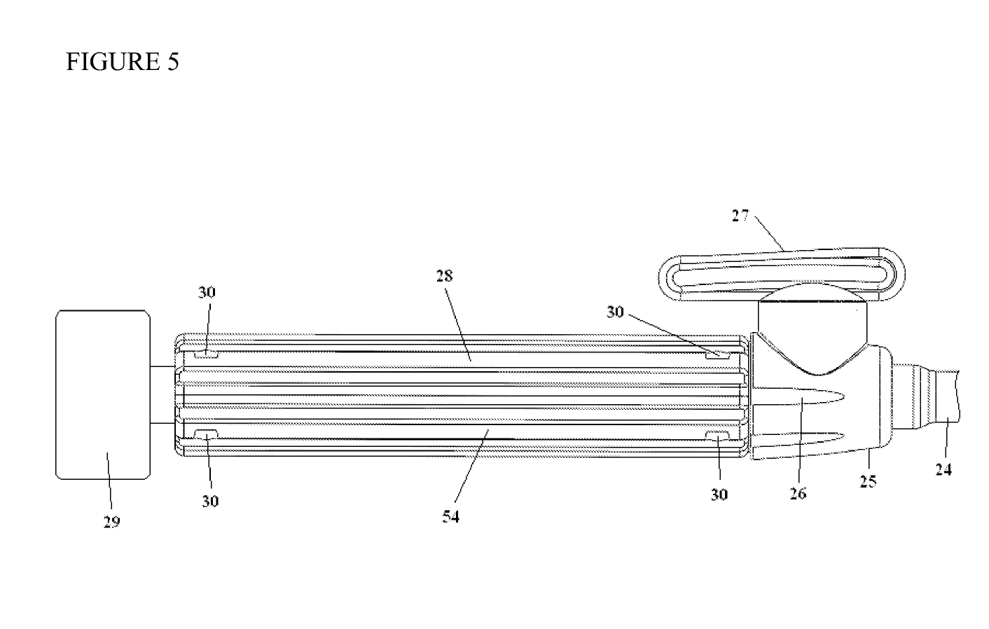

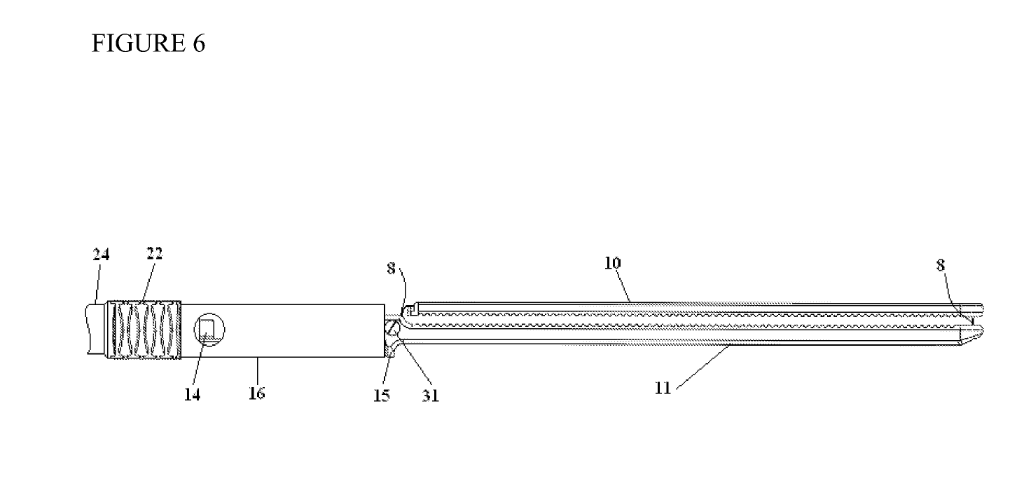

When combined edge to edge, FIGS. 5 and 6 schematically depict a profile view of a preferred embodiment of the surgery apparatus 400 in greater detail.

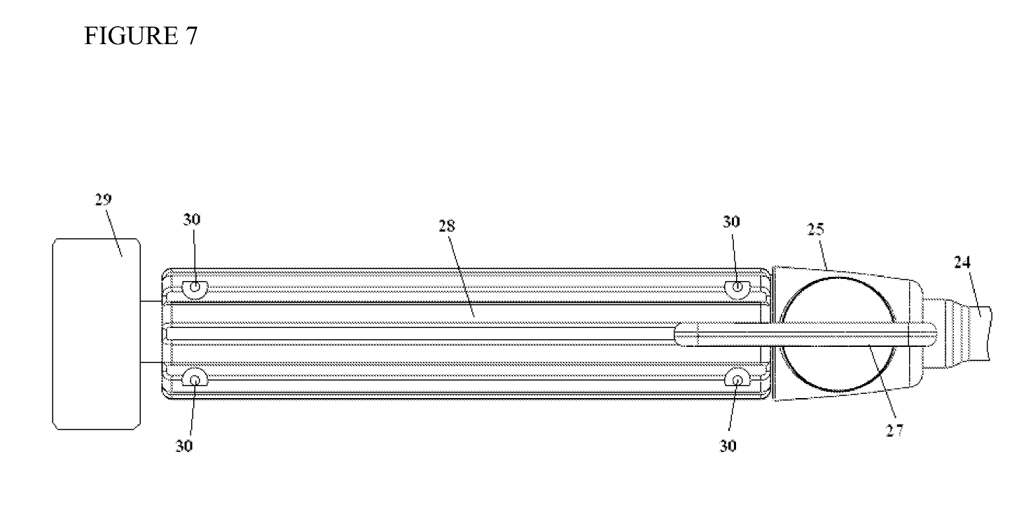

FIGS. 7 and 8 show additional details for an embodiment of the surgery apparatus, where the endoscopic shaft 24 of FIG. 7 is the endoscopic shaft 24 of FIG. 8, and the apparatus can be considered to comprise FIG. 7 placed to the left of FIG. 8.

FIG. 7 illustrates a cross section of an embodiment of the linear handle, with four peripherally placed handle bolt holes 30 to connect the male section 28 and female section (54 not shown) of the handle. The clamp actuation knob 29 at the handle end in this depiction can be turned to clamp the jaws by changing the motion or tension in cable 33. The endoscopic shaft 24 is shown to house the cable 33 shown in FIG. 8.

FIG. 8 illustrates internal details for an embodiment where the top jaw is actuated by a cable 33. By removing the bottom jaw sleeve 16 shown in FIG. 6 from the view of FIG. 8, the cable 33 is shown. The cable 33 is fastened by a clamp-end cable noose 34 and clamp-end crimp 56 to strap actuation mating-holes 32 in both ends of strap 8, and runs entirely throughout the surgery apparatus and clamp, terminating in a similar crimp and noose fashion to the clamp actuation knob 29 of the handle shown in FIG. 7 and FIG. 10. A key aspect of this embodiment is the passing of the cable 33 through the center of the flexible neck assembly 22 so that the articulation of the flexible neck assembly 22 has no affect on the tension of the cable 33 and thus no affect on the clamping force between the top jaw 10 and bottom jaw 11. The strap actuation mating-hole 32 couples the cable 33 to both ends of the strap 8 and allows for the transfer of power between the cable 33 and the top jaw 10. A support-bolt and nut 31 are fastened to a support-bolt hole 9 in the bottom jaw 11 and the bolt is enclosed by a spacer 35 and two washers 36 in order to provide retaining force and a reduced friction sliding path for the strap 8. The spacer and washers may be composed of Teflon. Also depicted are the elongated flexible transmission band assemblies 23 in mating contact with the bottom jaw mating posts 14, allowing for rotation of the bottom jaw 11.

FIG. 9 schematically illustrates one example of a handle consisting of a linear handle male section 28 and linear handle female section 54 which when coupled will internally provide the necessary linear handle threads 37.

FIG. 10 schematically illustrates an embodiment of the clamp actuation knob 29. The clamp actuation knob 29 can be divided into two sections: the distally located linear handle threads 38 and the proximally located twist-grip 39. The clamp actuation knob 29 is internally hollow to allow access to the cable 33. The proximally located twist-grip 39 may be externally textured by a twist-grip knurled surface in order to increase friction between hand and tool. In order to decrease friction between the clamp actuation knob 29 and the cable 33 an embedded bushing 40 may be located at the most proximal end of the cable 33 at the junction of handle-end cable noose 41, handle-end crimp 42 and cable 33. The bushing 40 may be composed of a variety of materials, including Teflon, rubber or metal. The bushing 40 provides holding force and acts as a reduced friction, rotation-allowing barrier between the cable 33 and the clamp actuation knob 29. The bushing 40 may also prevent the cable 33 from twisting along with the clamp actuation knob 29 when the twist-grip 39 is turned.

### EXAMPLE

### Clamping of the Kidney During a Laparoscopic Partial Nephrectomy

A laparoscopic partial nephrectomy is the removal of part of the kidney using minimally invasive surgical techniques, such as to remove a cancer along with a small amount of surrounding, normal tissue. Small incisions are made in the abdomen and the surgery is guided by a flexible videoscope, or laparoscope, inserted through one of the incisions. A gas is introduced within the abdominal cavity to enable better visualization of the kidney.

The surgery apparatus can be used to occlude blood flow to the portion of the kidney containing the cancer and is inserted through an abdominal incision using a trocar. The articulation assembly of the surgery apparatus is used to adjust the angulation of the clamp relative to the endoscopic shaft and handle according to the position of the kidney within the abdomen and the preferences of the surgeon. The clamp actuator assembly is used to close the jaws of the clamp of the surgery apparatus to exert sufficient pressure so as to occlude the blood flow to the portion of the kidney containing the cancer. The endoscopic shaft is then manipulated to provide an optimal view of the putative resection site, and the cancer is removed from the kidney with a margin of normal tissue. The blood supply around the resected area is closed to prevent bleeding as are entries into the collecting system. The clamp is then slowly released while still in place to allow gradual return of blood flow back into the area of resection while visualizing the resection bed. The clamp is immediately reapplied if further hemostasis or closure of the collecting system is warranted. Finally, the jaws of the surgery apparatus are opened fully and the clamp removed. If delayed bleeding occurs during the remainder of the procedure, the clamp can be rapidly reapplied to the kidney to provide temporary hemostasis. If significant bleeding continues despite best efforts when the clamp is removed, the operation can be converted to a standard “open” operation on the kidney leaving the clamp in place on the kidney so as to allow for a controlled entry into the abdomen rather than an emergent one.

We claim:

1. A laparoscopic surgical apparatus comprising: a handle section; a clamp formed by a top jaw and a bottom jaw to provide a pair of substantially parallel jaws, a laparoscopic section including an elongated cylindrical shaft having a proximal and a distal end, said proximal end being connected to said handle section and said distal end pivotally connected to said clamp, a clamp actuating assembly including a cable interconnecting said handle section and said clamp for actuating said clamp between an open position with said top jaw being displaced from said bottom jaw and a closed position state wherein said opposed parallel jaws in relatively closer positioned than when in said open position.

2. A surgical apparatus as recited in claim 1 wherein said handle section includes threaded connection to apply linear motion to move said cable and thereby relatively move said opposed jaws.

3. A surgical apparatus as recited in claim 2 wherein said threaded connection includes a hollow, threaded clamp actuation knob and said handle section includes mating threads with which to transfer power from said clamp actuation knob.

4. A surgical apparatus as recited in claim 3 wherein said cable is connected to said clamp actuation knob via a bushing.

5. A surgical apparatus as recited in claim 1 wherein said clamp further includes a strap cooperating with said opposed parallel jaws to cause the jaws to move between said open and closed positions while remaining substantially parallel.

6. A surgical apparatus as recited in claim 2 wherein said clamp further includes a strap cooperating with said opposed parallel jaws to cause the jaws to move between said open and closed positions while remaining substantially parallel.

7. A surgical apparatus as recited in claim 3 wherein said clamp further includes a strap cooperating with said opposed parallel jaws to cause the jaws to move between said open and closed positions while remaining substantially parallel.

8. A surgical apparatus as recited in claim 4 wherein said clamp further includes a strap cooperating with said opposed parallel jaws to cause the jaws to move between said open and closed positions while remaining substantially parallel.

9. A surgical apparatus as recited in claim 1 wherein said elongated cylindrical shaft defines a passage through which said cable passes, and further includes a flexible neck assembly to permit bending.

10. A surgical apparatus as recited in claim 2 wherein said elongated cylindrical shaft defines a passage through which said cable passes, and further includes a flexible neck assembly to permit bending.

11. A surgical apparatus as recited in claim 3 wherein said elongated cylindrical shaft defines a passage through which said cable passes, and further includes a flexible neck assembly to permit bending.

12. A surgical apparatus as recited in claim 4 wherein said elongated cylindrical shaft defines a passage through which said cable passes, and further includes a flexible neck assembly to permit bending.

13. A surgical apparatus as recited in claim 5 wherein said elongated cylindrical shaft defines a passage through which said cable passes, and further includes a flexible neck assembly to permit bending.

14. A surgical apparatus as recited in claim 6 wherein said elongated cylindrical shaft defines a passage through which said cable passes, and further includes a flexible neck assembly to permit bending.

15. A surgical apparatus as recited in claim 7 wherein said elongated cylindrical shaft defines a passage through which said cable passes, and further includes a flexible neck assembly to permit bending.

16. A surgical apparatus as recited in claim 8 wherein said elongated cylindrical shaft defines a passage through which said cable passes, and further includes a flexible neck assembly to permit bending.

17. A surgical apparatus as recited in claim 5 wherein said top jaw is fixed to said strap.

18. A surgical apparatus as recited in claim 6 wherein said top jaw is fixed to said strap.

19. A surgical apparatus as recited in claim 5 wherein said bottom jaw has a groove in which said strap may be slid.

20. A surgical apparatus as recited in claim 6 wherein said bottom jaw has a groove in which said strap may be slid.
